SEQUENCE LISTING (7 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 266 aa, Bacillus amyloliquefaciens
AQSVPYGVSQIKAPALHSQGYTGSNVKVAVIDSGIDSSHPALKVAGGASFVPSETNPFQDNNSHGTHVAGTVLAVAPSASLYAVKVLGADGSGQYSWIINGIEWAIANNMDVINMSLGGPSGSAALKAAVDKAVASGVVVVAAAGNEGTSGSSSTVGYPGKYPSVIAVGAVDSSNQRASFSSVGPELDVMAPGVSIWSTLPGNKYGAKSGTCMASPHVAGAAALILSKHPNWTNTQVRSSLENTTTKLGDSFYYGKGLINVEAAAQ

SEQ ID NO: 3 - protein, 274 aa, Bacillus licheniformis [sequence omitted]

SEQ ID NO: 4 - protein, 14 aa, Artificial Sequence
  Linker
GAATSGKLNGSTAG

SEQ ID NO: 5 - protein, 282 aa, Artificial Sequence
  circular permutation variant
GEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATGAATSGKLNGSTAGAQSVPWGISRVQAPAAHNRGLTGSGVKVAVLDTGISTHPDLNIRGGASFVP

SEQ ID NO: 6 - protein, 282 aa, Artificial Sequence
  circular permutation variant
YASLNGTSMATPHVAGAAALVKQKNPSWSNVQIRNHLKNTATSLGSTNLYGSGLVNAEAATGAATSGKLNGSTAGAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGST

SEQ ID NO: 7 - protein, 282 aa, Artificial Sequence
  construct for production of a permutation variant
LNGSTAGAQSVPWGISRVQAPAAHNRGLTGSGVKVAVLDTGISTHPDLNIRGGASFVPGEPSTQDGNGHGTHVAGTIAALNNSIGVLGVAPSAELYAVKVLGADGRGAISSIAQGLEWAGNNGMHVANLSLGSPSPSATLEQAVNSATSRGVLVVAASGNSGASSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATGAATSGK

(19) United States
(12) Patent Application Publication
(10) Pub. No.: US 2010/0196991 A1
(43) Pub. Date: Aug. 5, 2010

(54) NOVEL PROTEIN VARIANTS BY CIRCULAR PERMUTATION

(75) Inventors: Timothy O'Connell, Düsseldorf (DE); Karl-Heinz Maurer, Erkrath (DE); Nina Mußmann, Willich (DE); Susanne Wieland, Zons/Dormagen (DE)

Correspondence Address: CONNOLLY BOVE LODGE & HUTZ LLP, PO BOX 2207, WILMINGTON, DE, 19899-2207, US

(73) Assignee: Henkel AG & Co. KGaA, Dussedorf (DE)

(21) Appl. No.: 12/760,581

(22) Filed: Apr. 15, 2010

Related U.S. Application Data
(63) Continuation of application No. PCT/EP2008/062937, filed on Sep. 26, 2008.

(30) Foreign Application Priority Data
Oct. 16, 2007 (DE) 102007049830.8

(51) Int. Cl. C12N 9/56; C07H 21/04; C12N 15/63

(52) U.S. Cl. 435/222; 536/23.2; 435/320.1

(57) ABSTRACT

The present invention relates to a method for obtaining novel protein variants by circular permutation, and to the novel protein variants obtained by said method.

### RELATED APPLICATIONS

This application is a Continuation of Application PCT/EP2008/062937 filed on Sep. 26, 2008, which claims benefit of German application 102007049830.8, filed Oct. 16, 2007.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is Sequence_Listing_{—}13744_{—}00090_US. The size of the text file is 15 KB, and the text file was created on Apr. 12, 2010.

### BACKGROUND OF THE INVENTION

The present invention relates to a method for recovering new protein variants by circular permutation, and to the novel protein variants recovered by that method.

### DESCRIPTION OF RELATED ART

In order to optimize the properties of known washing-agent enzymes, it is usual to use mutation methods in which mutations are generated either in targeted fashion or by chance, or amino acids are introduced or removed by insertion or deletion.

Alternatives to conventional mutation methods are represented by so-called circular permutation methods. Circular permutation methods have been described in principle in the existing art for some time. Graf and Schachmann, for example (Proc. Natl. Acad. Sci. USA (1996) 93, 11591-11596), provide an overview of enzymes and other proteins with which circular permutation methods have been carried out.

In circular permutation methods, the N- and C-terminus of the mutated protein are redefined by genetic engineering. The basic assumption is that circular permutation methods can be carried out only with proteins in which the N- and C-terminus are located close to one another, so that the circular permutation leaves the overall structure of the protein substantially unchanged.

A further feature common to hitherto known enzymes and proteins with which permutation methods have been carried out is that the protein is expressed directly in active form. There also exist, however, groups of proteins, and in particular of enzymes, that are expressed in an inactive form as a preprotein, proprotein, or preproprotein, and only after expression are converted into the active form by enzymatic cleavage. The “pre” portion of the protein is a signal peptide that is responsible for guiding the expressed protein into the correct destination cell compartment, while the “pro” portion of the protein keeps the protein in inactive form until it is transformed into the active form, at the appropriate place and time, by activation at the target location.

It has hitherto been assumed that circular permutation methods can be carried out successfully only with proteins that on the one hand have N- and C-termini located close to one another, and on the other hand are expressed naturally with a mature amino acid sequence, i.e. without a pre-, pro-, and/or prepropeptide, since the localization of a pre-, pro-, and/or prepropeptide with reference to the mature protein was regarded as essential for correct processing and folding.

### BRIEF SUMMARY OF THE INVENTION

It has now been found, surprisingly and against all expectations, that even in the context of those proteins, and particularly enzymes, that are expressed naturally as a preprotein, proprotein, and/or preproprotein, novel protein variants can be obtained with the aid of the circular permutation method if the circular permutation method is carried out on the DNA of the mature protein, and if the DNA for the corresponding signal peptide, propeptide, and/or prepropeptide is appended to the DNA obtained by permutation.

This was surprising because it had not been expected on the one hand that the artificial construct would be successfully guided by the signal peptide into the intended target cell compartment, and on the other hand that folding of the protein would also successfully happen despite the location of the signal peptide and propeptide at the “wrong” site on the protein, and furthermore that activation of the protein would also occur as desired.

It has been discovered in particular according to the present invention, surprisingly, that by way of circular permutation methods on washing-agent enzymes, novel washing-agent enzymes which exhibit improved properties as compared with the initial molecules can be discovered.

### BRIEF DESCRIPTION OF THE DRAWINGS

The illustrations depict the results of the washing tests using the circular permutation variant NHT04240353 in accordance with Example 3.

FIG. 1 depicts the results of the washing experiments with blood/milk/ink, whole egg/carbon black, and chocolate milk/carbon black.

FIG. 2 depicts the results of the washing experiments with grass, milk/oil, cocoa, and once again blood/milk/ink. The results using the wild type (WT), i.e. BLAP, are set to 100% in each case.

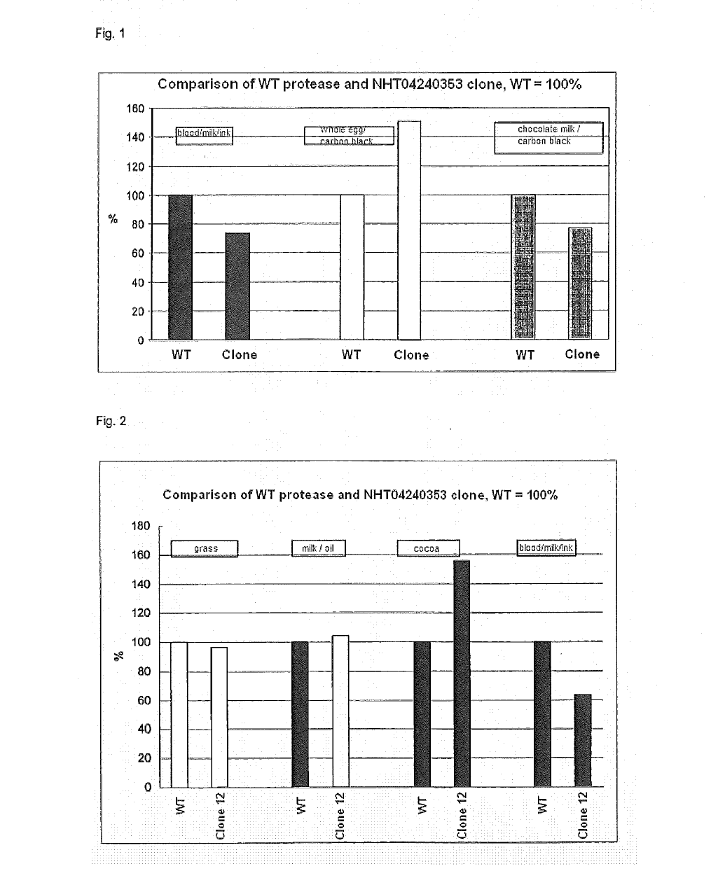

### DETAILED DESCRIPTION OF THE INVENTION

A first subject of the present invention is therefore a method for manufacturing the DNA coding for a circular permutation variant of the mature protein of a protein, in particular an enzyme, expressed naturally as a preprotein, proprotein, and/or preproprotein, wherein the DNA coding for the mature protein is subjected to a circular permutation method.

Also a subject of the present invention are therefore polynucleotides selected from

polynucleotides coding for circular permutation variants of the mature protein of proteins, in particular enzymes, expressed naturally as a preprotein, proprotein, and/or preproprotein,
mutants of polynucleotides in accordance with (a) having a sequence homology and/or sequence identity of at least 80, by preference at least 85 or 90%, particularly preferably at least 95, 98, or 99%, with respect to a polynucleotide in accordance with (a), wherein the sequence comparison refers in one embodiment to the circularly permuted polynucleotide without the polynucleotide coding for the bridging linker, and in another embodiment refers to the circularly permuted polynucleotide including the polynucleotide coding for the bridging linker,
mutants of polynucleotides in accordance with (a) having substitutions, insertions, deletions, or inversions of up to 50, 40, or 30, by preference up to 25, 20, or 15, particularly preferably up to 10, 9, 8, 7, or 6, especially up to 5, 4, 3, or 2 nucleotides, in particular having insertions or deletions of exactly one nucleotide, with reference to the polynucleotide sequence of a polynucleotide in accordance with (a), wherein the sequence comparison in one embodiment refers to the circularly permuted polynucleotide without the polynucleotide coding for the bridging linker, and in another embodiment refers to the circularly permuted polynucleotide including the polynucleotide coding for the bridging linker,
polynucleotides that encompass polynucleotides in accordance with (a), (b), or (c),
polynucleotides that code for circular permutation variants according to the present invention,
polynucleotides that comprise a sequence complementary to the polynucleotides in accordance with (a), (b), (c), (d), or (e).

In an embodiment preferred according to the present invention, polynucleotides according to the present invention code for circular permutation variants of BLAP selected from polypeptides having a sequence homology and/or sequence identity of at least 80%, by preference at least 85 or 90%, particularly preferably at least 95, 98 or 99%, with a polypeptide in accordance with SEQ ID NO: 5 or in accordance with SEQ ID NO: 6, and/or selected from polypeptides having insertions, deletions, or inversions of up to 30, 25, or 20, by preference up to 18, 16, 14, or 12, particularly preferably up to 10, 9, 8, 7, or 6, especially up to 5, 4, 3, or 2 amino acids, in particular having insertions or deletions of exactly one amino acid, with reference to a polypeptide having a polypeptide sequence in accordance with SEQ ID NO: 5 or in accordance with SEQ ID NO: 6.

The polynucleotides can exist as an individual strand or as a double strand. Also a subject of the invention, in addition to deoxyribonucleic acids, are the homologous and complementary ribonucleic acids.

Also a subject of the present invention are, in particular, those polynucleotides in which certain regions have been replaced, with consideration given to the differing codon usage of a host organism employed for expression, with other regions in order to enable expression of the polypeptide according to the present invention.

A further subject of the present invention is a method for manufacturing a circular permutation variant of the mature protein of a protein, in particular an enzyme, expressed naturally as a preprotein, proprotein, and/or preproprotein, encompassing the following steps:
a) carrying out a circular permutation method with the DNA of the mature protein,
b) incorporating the DNA obtained with the method in accordance with (a) into a suitable vector in the 3′ direction with respect to the DNA coding for a signal peptide and/or prepropeptide,
c) introducing the vector into a suitable host for expression of the DNA, and
d) if applicable, purifying the protein that is obtained.

A further subject of the present invention is therefore also polypeptides selected from
a) circular permutation variants that were obtained using a method according to the present invention, i.e. circular permutation variants of the mature protein of a protein expressed naturally as a preprotein, proprotein, and/or preproprotein,
b) variants of circular permutation variants in accordance with (a) having a sequence homology and/or sequence identity of at least 80%, preferably at least 85 or 90%, particularly preferably at least 95, 98, or 99%, with respect to the sequence of a circular permutation variant in accordance with (a), wherein the sequence comparison in one embodiment refers to the polypeptide sequence of the circular permutation variant without the sequence of the linker, and in another embodiment refers to the polypeptide sequence of the circular permutation variant including the sequence of the linker,
c) variants of circular permutation variants in accordance with (a) having substitutions, insertions, deletions, or inversions of up to 30, 25, or 20, by preference up to 18, 16, 14, or 12, particularly preferably up to 10, 9, 8, 7, or 6, especially up to 5, 4, 3, or 2 amino acids, in particular having insertions or deletions of exactly one amino acid, with reference to the polypeptide sequence of a polypeptide in accordance with (a) without the sequence of the linker, wherein the sequence comparison in one embodiment refers to the polypeptide sequence of the circular permutation variant without the sequence of the linker, and in another embodiment refers to the polypeptide sequence of the circular permutation variant including the sequence of the linker,
d) polypeptides that encompass the protein variants in accordance with (a), (b), or (c).

A “mature protein” is to be understood according to the present invention as the protein without consideration of the signal peptide and propeptide.

It may be imagined in principle that circular permutation, like other mutation methods, might have effects on various properties of the protein, for example on the stability of the protein or (in the case of enzymes) on activity or selectivity.

It has been found according to the present invention, surprisingly, that the washing performance of washing-agent enzymes with regard to specific stains can be considerably improved by circular permutation.

The circular permutation variants according to the present invention are therefore preferably the circular permutation variants of an enzyme, particularly preferably of a protease, especially of an alkaline protease of the subtilisin type.

Examples of alkaline proteases of the subtilisin type are the alkaline proteases from Bacillus lentus (BLAP) as well as the subtilisins BPN′ and Carlsberg, furthermore protease PB92, subtilisins 147 and 309, subtilisin DY, and the enzymes (to be classified, however, as subtilases and no longer as subtilisins in the strict sense) thermitase, proteinase K, and proteases TW3 and TW7. Subtilisin Carlsberg is obtainable in further developed form under the trade name ALCALASE® from Novozymes A/S, Bagsvaerd, Denmark. Subtilisins 147 and 309 are marketed by the Novozymes company under the trade names ESPERASE® and SAVINASE®, respectively. The variants marketed under the designation BLAP® are derived from the protease from Bacillus lentus DSM 5483 (WO 91/02792 A1) and are described in particular in WO 92/21760 A1, WO 95/23221 A1, WO 02/088340 A2, and WO 03/038082 A2. Further usable proteases from various strains of Bacillus species and B. gibsonii are evident from patent applications WO 03/054185, WO 03/056017, WO 03/055974, and WO 03/054184.

Other usable proteases are, for example, the enzymes obtainable under the trade names DURAZYM®, RELASE®, EVERLASE®, NAFIZYM, NATALASE®, KANNASE®, AND OVOZYMES® from the Novozymes company, under the trade names PURAFECT®, PURAFECT®OxP, and PROPERASE® from the Genencor company, under the trade name PROTOSOL® from Advanced Biochemicals Ltd., Thane, India, under the trade name WUXI® from Wuxi Snyder Bioproducts Ltd., China, under the trade names PROLEATHER® and PROTEASE P® from Amano Pharmaceuticals Ltd., Nagoya, Japan, and under the designation Proteinase K-16 from Kao Corp., Tokyo, Japan.

Circular permutation variants of these enzymes, as well as homologs thereof, by preference having the homology and identity values indicated previously, are preferably a subject according to the present invention.

The circularly permuted subtilisin is particularly preferably a circularly permuted BLAP protease having an initial sequence in accordance with SEQ ID NO: 1, a circularly permuted BPN′ protease having an initial sequence in accordance with SEQ ID NO: 2, or a circularly permuted subtilisin Carlsberg having an initial sequence in accordance with SEQ ID NO: 3, and homologs thereof.

In the circular permutation variants according to the present invention, the original termini of the mature initial enzyme are linked to one another by preference by an arbitrary linker of a length from 1 to 40, particularly preferably 5 to 30, in particular 10 to 20 amino acids. A linker having a length from 10 to 20 amino acids, particularly preferably a linker having 12 to 16 amino acids, especially a linker having 13, 14, or 15 amino acids, in particular the linker GAATSGKLNGSTAG (SEQ ID NO: 4), is used by preference to manufacture the circularly permuted subtilisins.

The linker can in principle be a oligopeptide or polypeptide having any sequence. Care must be taken, if applicable, that the sequence of the linker is selected so that the linker possesses sufficient flexibility to link the two original termini of the initial protein to one another. The aforementioned linker having the sequence GAATSGKLNGSTAG (SEQ ID NO: 4), for example, has proven suitable for bridging the BLAP proteases. In principle, however, any other desired linker can of course also be used to link the original termini. The sequence of the linker is of subordinate important for the function of an enzyme because it is located far away from the active center of the enzyme.

The new termini of the circular permutation variant are by preference at a distance of at least 10 amino acids, particularly preferably at least 20 amino acids, in particular at least 30, 35, or 40 amino acids, from the original termini.

In a preferred embodiment, the new termini of the circular permutation variants are located outside the actual tertiary structure, in particular in the region of loop structures of the initial enzyme, i.e. in regions that lie outside the complex folding of the protein and instead exhibit bridging properties.

In this embodiment the termini of the circularly permuted BLAP protease are therefore located between amino acids 1-4, 10-14, 18-26, 32-45, 49-62, 70-76, 78-87, 93-102, 115-118, 122-131, 142-146, 154-169, 175-179, 181-183, 185-192, 196-199, 203-207, 211-215, 231-237, 247-264, or 267-268 of the initial enzyme.

In this embodiment the termini of the circularly permuted BPN′ protease are therefore correspondingly located between amino acids 1-7, 10-14, 19-26, 32-46, 50-64, 95-104, 117-120, 124-133, 145-148, 156-175, 181-185, 187-189, 191-198, 202-205, 209-213, 217-220, 237-243, 252-270, or 273-275 of the initial enzyme.

In this embodiment the termini of the circularly permuted subtilisin Carlsberg are therefore correspondingly located between amino acids 1-7, 9-13, 19-26, 32-46, 50-64, 72-89, 95-104, 116-121, 124-134, 145-148, 156-175, 181-185, 187-189, 191-198, 202-205, 209-213, 217-220, 237-243, 250-270, or 274-275 of the initial enzyme.

It has, however, been found according to the present invention, surprisingly, that localization of the new termini in a loop structure or in a similar region located outside the actual tertiary structure of the initial enzyme is not necessary for the functionality of the enzyme, but that it is also possible for the new termini to be localized in the midst of the tertiary and/or quaternary structure of the folded initial protein without disrupting the activity or stability of the enzyme. In this embodiment, the termini of the circularly permuted protein are therefore not located in the region of loop structures.

In this embodiment the termini of the circularly permuted BLAP protease are therefore located between amino acids 4-10, 14-18, 26-32, 45-49, 62-70, 76-78, 87-93, 102-115, 118-122, 131-142, 146-154, 169-175, 179-181, 183-185, 192-196, 199-203, 207-211, 215-231, 237-247, 264-267, or 268-end of the initial enzyme.

In this embodiment the termini of the circularly permuted BPN′ protease are therefore correspondingly located between amino acids 7-10, 14-19, 26-32, 46-50, 64-95, 104-117, 120-124, 133-145, 148-156, 175-181, 185-187, 189-191, 198-202, 205-209, 213-217, 220-237, 243-252, 270-273, or 275-end of the initial enzyme.

In this embodiment the termini of the circularly permuted subtilisin Carlsberg are therefore correspondingly located between amino acids 7-9, 13-19, 26-32, 46-50, 64-72, 89-95, 104-116, 121-124, 134-145, 148-156, 175-181, 185-187, 189-191, 198-202, 205-209, 213-217, 220-237, 243-250, 270-274, or 275-end of the initial enzyme.

In an embodiment particularly preferred according to the present invention, the circular permutation variants of BLAP are selected from polypeptides having a sequence homology and/or sequence identity of at least 80%, by preference at least 85 or 90%, particularly preferably at least 95, 98, or 99%, with a polypeptide in accordance with SEQ ID NO: 5 or in accordance with SEQ ID NO: 6, and/or from polypeptides having insertions, deletions, or inversions of up to 30, 25, or 20, by preference up to 18, 16, 14, or 12, particularly preferably up to 10, 9, 8, 7, or 6, especially up to 5, 4, 3, or 2 amino acids, in particular having insertions or deletions of exactly one amino acid, with reference to a polypeptide having a polypeptide sequence in accordance with SEQ ID NO: 5 or in accordance with SEQ ID NO: 6.

The measure of homology is a percentage identity, as can be determined e.g. according to the method indicated by D. J. Lipman and W. R. Pearson in Science 227 (1985), pp. 1435-1441. This indication can refer to the entire protein or to the region to be respectively allocated. A further construed homology term, “similarity,” also brings into consideration conserved variations, i.e. amino acids having a similar chemical activity, since they usually exert similar chemical activities within the protein. For nucleic acids, only the percentage identity is known. According to the present invention, the indication to be taken as a basis for identity or homology is the complete sequence of the particular polypeptide (or polynucleotide) according to the present invention that is being considered, or the complete sequence of the polypeptide region (or polynucleotide region) according to the present invention that is being considered, and not simply the respectively overlapping region between the polypeptide (or polynucleotide) according to the present invention or polypeptide region (or polynucleotide region) according to the present invention and a comparison protein (or comparison nucleotide). If the indications as to homology and identity refer to a polypeptide according to the present invention exclusive of the linker peptide, the polypeptide sequence that is bridged by the linker is to be regarded as a continuous sequence. This likewise applies analogously to the polynucleotide sequence coding for a polypeptide according to the present invention.

A further subject of the present invention is vectors, in particular cloning vectors and expression vectors, that contain polynucleotides according to the present invention, as well as cells, in particular host cells, that contain polynucleotides, vectors, and/or polypeptides according to the present invention.

In a preferred embodiment, the cells are selected from Gram-negative bacteria, in particular those of the species Escherichia coli or Klebsiella, in particular from the strains of E. coli K12, E. coli B, or Klebsiella planticola, and very particularly from derivatives of the strains Escherichia coli BL21 (DE3), E. coli RV308, E. coli DH5α, E. coli JM 109, E. coli XL-1, or Klebsiella planticola (Rf)

In a further preferred embodiment, the cells are selected from Gram-positive bacteria, in particular those of the genera Bacillus, Staphylococcus or Corynebacteria, very particularly of the species Bacillus lentus, B. lichenifonnis, B. amyloliquefaciens, B. subtilis, B. globigii, B. gibsonii, B. pumilus, or B. alcalophilus, Staphylococcus carnosus, or Corynebacterium glutamicum.

Agents containing polypeptides according to the present invention.

A further subject of the invention is represented by agents that contain aforementioned polypeptides according to the present invention.

All types of agents, in particular mixtures, formulations, solutions, etc., whose usability is improved by addition of an above-described protein according to the present invention, are herewith included in the range of protection of the present invention. These can be, depending on the area of application, for example solid mixtures, for example powders having freeze-dried or encapsulated proteins, or gelled or liquid agents. Preferred formulations contain, for example, buffer substances, stabilizers, reaction partners, and/or cofactors of the proteases, and/or other ingredients synergistic with the proteases. Particularly to be understood thereamong are agents for the areas of application set forth hereinafter. Further areas of application are evident from the existing art.

Possible areas of application in this context are, in particular, utilization in order to recover or treat raw materials or intermediate products in textile manufacturing, in particular for the removal of protective layers on fabrics, in particular on wool or silk, as well as utilization for the care of textiles that contain natural fibers, in particular wool or silk.

Also correspondingly a subject of the invention are methods for treating textile raw materials and for textile care, in which polypeptides according to the present invention are used in at least one of the method steps. Preferred thereamong are methods for textile raw materials, fibers, or textiles having natural constituents, in particular for those having wool or silk. These can be, for example, methods in which materials are prepared for processing into textiles, for example for anti-felting finishing; or, for example, methods that supplement the cleaning of previously worn textiles with a care-providing component.

Further possible areas of application are, for example:

utilization for biochemical analysis or for the synthesis of low-molecular-weight compounds or of proteins; preferred thereamong is utilization for determining terminal groups in the context of peptide sequence analysis;
utilization for the preparation, purification, or synthesis of natural substances or biological useful substances;
utilization for the treatment of natural raw materials, in particular for surface treatment, very particularly in a method for treating leather, in particular for dehairing leather;
utilization for the treatment of photographic films, in particular for removing gelatin-containing or similar protective layers; and utilization for the manufacture of foods or animal feeds, in particular for enzymatic treatment of soy milk and/or soy milk products.

Use of the aforementioned polypeptides according to the present invention in all further technical fields for which said use proves to be suitable is incorporated in principle into the range of protection of the present Application.

A further utilization possibility according to the present invention is use of the polypeptides according to the present invention in cosmetic agents. All types of cleaning and care-providing agents for human skin or human hair are to be understood thereamong, in particular cleaning agents. Depending on the intended application, the agent can also be a pharmaceutical agent.

Shampoos, soaps, washing lotions, creams, peels, as well as oral, dental, or dental-prosthesis care-providing agents may be recited as examples of cosmetic and/or pharmaceutical agents according to the present invention. These agents can also, in particular, contain constituents such as those recited hereinafter for washing and cleaning agents.

A subject that is particularly preferred according to the present invention is represented by washing and cleaning agents that contain polypeptides according to the present invention. This is because, as shown in the exemplifying embodiments of the present Application, it has been possible, surprisingly, to ascertain for washing and cleaning agents having a protease preferred according to the present invention an increase in washing performance as compared with agents having proteases used in conventional fashion.

The “washing performance” or “cleaning performance” of a washing agent or cleaning agent, respectively, is to be understood for purposes of the present Application as the effect that the agent in question exerts on the soiled items, for example textiles or objects having hard surfaces. Individual components of such agents, in particular the enzymes according to the present invention, are assessed in terms of their contribution to the washing or cleaning performance of the entire washing or cleaning agent, respectively. Particular consideration must be given in this context to the fact that the enzymatic properties of an enzyme do not immediately allow a conclusion as to its contribution to the washing performance of an agent. To the contrary, in addition to enzymatic activity, factors especially such as stability, substrate binding, binding to the material being cleaned, or interactions with other ingredients of the washing or cleaning agent also play a role here, in particular including possible synergistic effects in the context of stain removal.

A further subject of the present invention is therefore washing and cleaning agents, in particular those containing surfactant and/or bleaching agent, that contain a polypeptide according to the present invention.

The washing and cleaning agents according to the present invention can involve all conceivable types of cleaning agents, both concentrates and agents to be applied undiluted, for use on a commercial scale, in a washing machine or for hand laundering or cleaning. Included thereamong are, for example, washing agents for textiles, carpets, or natural fibers for which the term “washing agent” is used in accordance with the present invention. Also included thereamong are, for example, dishwashing agents for automatic dishwashers, or manual dishwashing agents, or cleaners for hard surfaces such as metal, glass, porcelain, ceramic, tiles, stone, painted surfaces, plastics, wood, or leather; the term “cleaning agent” is used for these in accordance with the present invention. Further to be regarded as washing and cleaning agents for purposes of the present invention are sterilizing and disinfecting agents.

Embodiments of the present invention encompass all administration forms of the washing or cleaning agents according to the present invention that are appropriate and/or are established according to the existing art. Included thereamong are, for example, solid, powdered, liquid, gelled, or pasty agents, optionally also made up of multiple phases, compressed or not compressed; also included thereamong are, for example, extrudates, granulates, tablets, or pouches, packaged both in large containers and in portions.

In addition to a polypeptide according to the present invention, a washing or cleaning agent according to the present invention optionally contains further ingredients such as further enzymes, enzyme stabilizers, surfactants, e.g. nonionic, anionic, and/or amphoteric surfactants, bleaching agents, bleach activators, bleach catalysts, builders, solvents, thickeners, sequestering agents, electrolytes, optical brighteners, anti-gray agents, silver corrosion inhibitors, color transfer inhibitors, foam inhibitors, abrasive substances, dyes, scents, antimicrobial active substances, UV absorbers, so-called soil release active substances or soil repellents, and further usual ingredients as applicable. With regard to ingredients of the aforementioned groups usable according to the present invention, reference is made in particular to the Application DE 10 2007 049 830.8.

Surfactants are contained in cleaning or washing agents according to the present invention by preference in a total quantity from 5 to 50 wt %, particularly preferably from 8 to 30 wt %, based on the completed agent; in a preferred embodiment, at least one nonionic surfactant is utilized.

The bleaching-agent content of the washing or cleaning agents can be 1 to 40 wt % and in particular 10 to 20 wt %, perborate monohydrate or percarbonate advantageously being used as a bleaching agent.

Builder substances can be contained in the washing or cleaning agents according to the present invention, if applicable, in quantities of up to 90 wt %. They are preferably contained in quantities of up to 75 wt %.

Thickeners are contained in the agents according to the present invention by preference in a quantity of up to 5 wt %, in particular from 0.05 to 2 wt %, and particularly preferably from 0.1 to 1.5 wt %, based on the completed composition.

In order to increase washing and cleaning performance, agents according to the present invention can contain further enzymes in addition to the polypeptides according to the present invention, all enzymes established in the existing art for these purposes being usable in principle. These include, in particular, further proteases, amylases, lipases, hemicellulases, cellulases, or oxidoreductases, as well as preferably mixtures thereof. These enzymes are, in principle, of natural origin; improved variants based on the natural molecules are available for use in washing and cleaning agents and are correspondingly preferred for use. Agents according to the present invention contain these further enzymes by preference in total quantities from 1×10⁻⁶ to 5 percent by weight, based on active protein.

Preferred among the further proteases are the natural forms of the enzymes of the subtilisin type that have already been described previously, as well as further washing-agent proteases that also have already been described previously.

Examples thereof are the enzymes already recited above: the alkaline protease from Bacillus lentus, subtilisins BPN′ and Carlsberg, protease PB92, subtilisins 147 and 309, subtilisin DY, and the enzymes (to be classified, however, as subtilases and no longer as subtilisins in the narrower sense) thermitase, proteinase K, and proteases TW3 and TW7. Subtilisin Carlsberg is obtainable in further developed form under the trade name ALCALASE® from Novozymes A/S, Bagsvaerd, Denmark. Subtilisins 147 and 309 are marketed by the Novozymes company under the trade names ESPERASE® and SAVINASE®, respectively. The variants marketed under the designation BLAP® are derived from the protease from Bacillus lentus DSM 5483 (WO 91/02792 A1) and are described in particular in WO 92/21760 A1, WO 95/23221 A1, WO 02/088340 A2, and WO 03/038082 A2. Further usable proteases from various strains of Bacillus species and B. gibsonii are evident from patent applications WO 03/054185, WO 03/056017, WO 03/055974, and WO 03/054184.

Other usable proteases are, for example, the enzymes obtainable under the trade names DURAZYM®, RELASE®, EVERLASE®, NAFIZYM, NATALASE®, KANNASE®, AND OVOZYMES® from the Novozymes company, under the trade names PURAFECT®, PURAFECT® OxP, and PROPERASE® from the Genencor company, under the trade name PROTOSOL® from Advanced Biochemicals Ltd., Thane, India, under the trade name WUXI® from Wuxi Snyder Bioproducts Ltd., China, under the trade names PROLEATHER® and PROTEASE P® from Amano Pharmaceuticals Ltd., Nagoya, Japan, and under the designation Proteinase K-16 from Kao Corp., Tokyo, Japan.

Examples of amylases usable according to the present invention are the α-amylases from Bacillus licheniformis, from B. amyloliquefaciens, or from B. stearothermophilus, and their further developments improved for use in washing and cleaning agents. The enzyme from B. licheniformus is available from the Novozymes company under the name Termamyl®, and from the Genencor company under the name PURASTAR® ST. Further developed products of these α-amylases are available from the Novozymes company under the trade names DURAMYL® and TERMAMYL® ultra, from the Genencor company under the name PURASTAR® OxAm, and from Daiwa Seiko Inc., Tokyo, Japan, as KEISTASE®. The α-amylase from B. amyloliquefaciens is marketed by the Novozymes company under the name BAN®, and derived variants of the α-amylase from B. stearothermophilus are marketed, again by the Novozymes company, under the names BSG® and NOVAMYL®. Further usable commercial products are, for example, AMYLASE-LT® and STAINZYME®, the latter also from the Novozymes company.

Also to be emphasized for this purpose are the α-amylase from Bacillus sp. A 7-7 (DSM 12368) disclosed in Application WO 02/10356 A2 and the cyclodextrin-glucanotransferase (CGTase) from B. agaradherens (DSM 9948) described in Application WO 02/44350 A2. Also usable are the amylolytic enzymes that belong the sequence space of α-amylases and are defined in Application WO 03/002711 A2, and those described in Application WO 03/054177 A2. Fusion products of the aforesaid molecules are likewise usable, for example those from Application DE 10138753 A1.

The further developments of the α-amylase from Aspergillus niger and A. oryzae, obtainable from the Novozymes company under the trade names FUNGAMYL®, are also suitable. A further commercial product is, for example, AMYLASE-LT®.

Agents according to the present invention can contain lipases or cutinases, in particular because of their triglyceride-cleaving activities but also in order to generate peracids in situ from suitable precursors. These include, for example, the lipases obtainable originally from Humicola lanuginosa (Thermomyces lanuginosus) or further-developed lipases, in particular those having the D96L amino acid exchange. They are marketed, for example, by the Novozymes company under the trade names LIPOLASE®, LIPOLASE® ULTRA, LIPOPRIME®, LIPOZYME®, and LIPEX®. The cutinases that were originally isolated from Fusarium solani pisi and Humicola insolens are moreover usable, for example. Additional usable lipases are obtainable from the Amano company under the designations LIPASE CE®, LIPASE P®, LIPASE B®, OR LIPASE CES®, LIPASE AKG®, BACILLIS SP. LIPASE®, LIPASE AP®, LIPASE M-AP®, and LIPASE AML®. The lipases and cutinases from, for example, the Genencor company, whose starting enzymes were originally isolated from Pseudomonas mendocina and Fusarium solanii, are usable. To be mentioned as further important commercial products are the preparations M1 LIPASE® and LIPOMAX® originally marketed by the Gist-Brocades company, and the enzymes marketed by Meito Sangyo KK, Japan, under the names LIPASE MY-30®, LIPASE OF®, and LIPASE PL®, as well as the LUMAFAST® product of the Genencor company.

Agents according to the present invention can, especially if they are intended for the treatment of textiles, contain cellulases, depending on the purpose as pure enzymes, as enzyme preparations, or in the form of mixtures in which the individual components advantageously complement one another in terms of their various performance aspects. These performance aspects include, in particular, contributions to primary washing performance, the secondary washing performance of the agent (anti-redeposition effect or graying inhibition), and avivage (fabric effect), or even exertion of a “stone-washed” effect.

A usable fungus-based cellulase preparation rich in endoglucanase (EG), and its further developments, are offered by the Novozymes company under the trade name CELLUZYME®. The products ENDOLASE® and CAREZYME®, likewise obtainable from the Novozymes company, are based on the 50 kD EG and 43 kD EG, respectively, from H. insolens DSM 1800. Further usable commercial products of this company are CELLUSOFT® and RENOZYME®. The latter is based on application WO 96/29397 A1. Improved-performance cellulase variants are described, for example, in Application WO 98/12307 A1. The cellulases disclosed in Application WO 97/14804 A1 are also usable, for example the 20 kD EG from Melanocarpus disclosed therein that is available from the AB Enzymes company, Finland, under the trade names ECOSTONE® and BIOTOUCH®. Other commercial products of the AB Enzymes company are ECONASE® and ECOPULP®. Other suitable cellulases from Bacillus sp. CBS 670.93 and CBS 669.93 are disclosed in WO 96/34092 A2, the one from Bacillus sp. CBS 670.93 being obtainable from the Genencor company under the trade name PURADAX®. Other commercial products of the Genencor company are “Genencor detergent cellulase L” and INDIAGE® Neutra.

In particular in order to remove certain problem stains, agents according to the present invention can contain, alongside polypeptides according to the present invention, further enzymes that are grouped under the term “hemicellulases.” These include, for example, mannanases, xanthanlyases, pectinlyases (=pectinases), pectinesterases, pectatelyases, xyloglucanases (=xylanases), pullulanases, and (β-glucanases. Suitable mannanases are obtainable, for example, under the names GAMANASE® and PEKTINEX AR® from the Novozymes company, under the name ROHAPEC® B1L from the AB Enzymes company, and under the name Pyrolase® from Diversa Corp., San Diego, Calif., USA. A suitable β-glucanase from a B. alcalophilus is evident, for example, from Application WO 99/06573 A1. The β-glucanase recovered from B. subtilis is available under the name CEREFLO® from the Novozymes company.

To enhance the bleaching effect, washing and cleaning agents according to the present invention can contain oxidoreductases, for example oxidases, oxygenases, catalases, peroxidases such as halo-, chloro-, bromo-, lignin, glucose, or manganese peroxidases, dioxygenases, or laccases (phenoloxidases, polyphenoloxidases). Suitable commercial products that may be mentioned are DENILITE® 1 and 2 of the Novozymes company. Advantageously, preferably organic, particularly preferably aromatic compounds that interact with the enzymes are additionally added in order to enhance the activity of the relevant oxidoreductases (enhancers) or, if there is a large difference in redox potentials between the oxidizing enzymes and the stains, to ensure electron flow (mediators).

The polypeptides according to the present invention, as well as the enyzmes used additionally, can be added to agents according to the present invention in any form established in accordance with the existing art. Included thereamong are, for example, the solid preparations obtained by granulation, extrusion, or lyophilization or, especially in the case of liquid or gelled agents, solutions of the enzymes, advantageously as concentrated as possible, low in water, and/or with stabilizers added.

Alternatively, these proteins can be encapsulated for both the solid and the liquid administration form, for example by spray-drying or extruding the enzyme solution together with a preferably natural polymer, or in the form of capsules, for example those in which the enzymes are enclosed e.g. in a solidified gel, or in those of the core-shell type, in which an enzyme-containing core is covered with a protective layer impermeable to water, air, and/or chemicals. Further active substances, for example stabilizers, emulsifiers, pigments, bleaching agents, or dyes, can additionally be applied in superimposed layers. Such capsules are applied in accordance with methods known per se, for example by vibratory or rolling granulation or in fluidized bed processes. Such granulates are advantageously low in dust, e.g. as a result of the application of polymeric film-forming agents, and are stable in storage thanks to the coating.

It is furthermore possible to package two or more enzymes together, for example a polypeptide according to the present invention and a further enzyme, so that a single granulate exhibits several enzyme activities.

A protein contained in an agent according to the present invention, in particular including the polypeptide according to the present invention, can be protected, especially during storage, from damage such as, for example, inactivation, denaturing, or decomposition, e.g. resulting from physical influences, oxidation, or proteolytic cleavage. An inhibition of proteolysis is particularly preferred in the context of microbial recovery of the proteins and/or enzymes, in particular when the agents also contain proteases. Preferred agents according to the present invention can contain stabilizers for this purpose.

Reversible protease inhibitors are one group of stabilizers. Benzamidine hydrochloride, borax, boric acids, boronic acids, or salts or esters thereof are often used for this, among them principally derivatives having aromatic groups, e.g. ortho-, meta-, or para-substituted phenylboronic acids, in particular 4-formylphenylboronic acid, or the respective salts or esters of the aforesaid compounds. Peptide aldehydes, i.e. oligopeptides having a reduced C-terminus, in particular those made up of 2 to 50 monomers, are also used for this purpose. Ovomucoid and leupeptin, among others, are among the peptide-type reversible protease inhibitors. Specific reversible peptide inhibitors for the protease subtilisin, as well as fusion proteins of proteases and specific protease inhibitors, are also suitable for this.

Further enzyme stabilizers are aminoalcohols such as mono-, di-, triethanol- and -propanolamine and mixtures thereof, aliphatic carboxylic acids up to C₁₂ such as, for example, succinic acid, other dicarboxylic acids, or salts of the aforesaid acids. End-capped fatty acid amide alkoxylates are also suitable for this purpose. Certain organic acids used as builders are additionally capable, as disclosed in WO 97/18287, of stabilizing a contained enzyme.

Lower aliphatic alcohols, but principally polyols, for example glycerol, ethylene glycol, propylene glycol, or sorbitol, are other frequently used enzyme stabilizers. Diglycerol phosphate also protects against denaturing due to physical influences. Salts of calcium and/or magnesium are likewise used, for example calcium acetate or calcium formate.

Polyamide oligomers or polymeric compounds such as lignin, water-soluble vinyl copolymers or cellulose ethers, acrylic polymers, and/or polyamides stabilize the enzyme preparation, inter alia with respect to physical influences or pH fluctuations. Polyamine-N-oxide-containing polymers act simultaneously as enzyme stabilizers and as color transfer inhibitors. Other polymeric stabilizers are the linear C₈ to C₁₈ polyoxyalkylenes. Alkyl polyglycosides can also stabilize the enzymatic components of the agent according to the present invention, and by preference are capable of additionally increasing its performance. Crosslinked nitrogen-containing compounds by preference perform a dual function as soil release agents and as enzyme stabilizers. Hydrophobic nonionic polymer stabilizes, in particular, a cellulase that may be contained.

Reducing agents and antioxidants enhance the stability of the enzymes with respect to oxidative breakdown; sulfur-containing reducing agents, for example, are common for this purpose. Other examples are sodium sulfite and reducing sugars.

It is particularly preferred to use combinations of stabilizers, for example made up of polyols, boric acid and/or borax, the combination of boric acid or borate, reducing salts, and succinic acid or other dicarboxylic acids, or the combination of boric acid or borate with polyols or polyamino compounds and with reducing salts. The effect of peptide aldehyde stabilizers is favorably enhanced by combination with boric acid and/or boric acid derivatives and polyols, and even further by the additional action of divalent cations such as, for example, calcium ions.

Because agents according to the present invention can be offered in all conceivable forms, polypeptides according to the present invention in all formulations appropriate for addition to the respective agents represent respective embodiments of the present invention. These include, for example, liquid formulations, solid granules, or capsules.

The encapsulated form is a good choice for protecting the enzymes or other ingredients from other constituents such as, for example, bleaching agents, or for permitting controlled release. Depending on the size of these capsules, a distinction is made among micro-, and nanocapsules, microcapsules being particularly preferred for enzymes. Such capsules are disclosed, for example, by Patent Applications WO 97/24177 and DE 19918267. One possible encapsulation method involves encapsulating the proteins, starting from a mixture of the protein solution with a solution or suspension of starch or a starch derivative, in that substance. An encapsulating method of this kind is described by Application WO 01/38471.

In the case of solid agents, the proteins—polypeptides according to the present invention as well as further enzymes that may optionally be contained—can be used e.g. in dried, granulated, and/or encapsulated form. They can be added separately, i.e. as an independent phase, or together with other constituents in the same phase, with or without compacting. If microencapsulated enzymes are to be processed in solid form, water can be removed from the aqueous solutions resulting from preparation using methods known from the existing art, such as spray drying, centrifuging, or resolubilization. The particles obtained in this fashion usually have a particle size between 50 and 200 μm.

The proteins proceeding from a protein recovery and preparation process carried out according to the existing art can be added to liquid, gelled, or pasty agents according to the present invention in concentrated aqueous or nonaqueous solution, suspension, or emulsion, but also in gel form or encapsulated form or as a dried powder. Washing or cleaning agents of this kind according to the present invention are usually produced by simple mixing of the ingredients, which can be introduced, in the mass or as a solution, into an automatic mixer.

A cleaning agent according to the present invention, in particular a cleaner according to the present invention for hard surfaces, can contain one or more Propellants (per INCI nomenclature), usually in a quantity from 1 to 80 wt %, by preference 1.5 to 30 wt %, in particular 2 to 10 wt %, particularly preferably 2.5 to 8 wt %, extremely preferably 3 to 6 wt %.

A particular embodiment according to the present invention of the present invention is represented by the use of a polypeptide according to the present invention to activate, deactivate, or release ingredients of washing or cleaning agents.

A particular subject of the invention is further represented by methods for cleaning textiles or hard surfaces in which a polypeptide according to the present invention is used in at least one of the method steps.

Included thereamong are both manual and automatic methods, automatic methods being preferred because of their more precise controllability with regard, for example, to the quantities and contact times used.

A further subject of the invention is represented by the use of an alkaline protease according to the present invention for the cleaning of textiles or hard surfaces.

A further subject of the present invention is also a product containing a composition according to the present invention or a washing or cleaning agent according to the present invention, in particular a cleaner according to the present invention for hard surfaces, and a spray dispenser. The product can in this context be both a single-chamber and a multi-chamber vessel, in particular a two-chamber vessel. In this context, the spray dispenser is preferably a manually activated spray dispenser, selected in particular from the group encompassing aerosol spray dispensers (pressurized-gas containers, also referred to inter alia as a spray can), spray dispensers that themselves build up pressure, pump spray dispensers, and trigger spray dispensers, in particular pump spray dispensers and trigger spray dispensers having a container made of transparent polyethylene or polyethylene terephthalate. Spray dispensers are described more exhaustively in WO 96/04940 (Procter & Gamble) and in the U.S. patents cited therein regarding spray dispensers, to which patents in their entirety reference is made in this regard, and the content of which is hereby incorporated into this Application. Trigger spray dispensers and pump atomizers possess the advantage, as compared with pressurized-gas containers, that no propellant needs to be used. By means of suitable particle-capable attachments, nozzles, etc. (so-called “nozzle valves”) on the spray dispenser, in this embodiment an enzyme that may be contained can optionally also be added to the agent in a form immobilized on particles, and thus metered as a cleaning foam.

The Examples that follow explain the invention further without limiting it thereto.

### EXEMPLIFYING EMBODIMENTS

### Example 1

### Carrying Out the Circular Permutation

The gene for a mature protease is flanked on both sides by the fragments of the DNA for a variable linker (P linker; length 1 to 40 amino acids), and the construct thereby obtained is cloned into a plasmid (initial plasmid). Located at the 5′ end flanking in the 5′ direction, and at the 3′ end flanking in the 3′ direction, of the two DNA fragments coding for the P linker are restriction enzyme interfaces that, following a restriction digest, form mutually fitting overhangs. The two DNA fragments flanking the gene of the mature protein and coding for the P linker can thus, after the restriction digest, ligate with one another and thus also link to one another the nucleic acids coding for the C- and the N-terminus of the mature protein. For this purpose, the construct obtained by restriction digest is closed into a ring using a ligase. The length of the P linker is selected so that the distance between the C- and N-terminus in the native protein can be spanned.

After ligation of the DNA coding for the C- and N-termini, the ring is then opened enzymatically at a random location, thus yielding a nucleic acid that codes for a mature protease having new C- and N-termini. Those nucleic acids obtained by circular permutation that code for an active protease having properties to the extent possible can then be identified by means of a screening method using a screening plasmid in a suitable host.

The nucleic acid coding for the prepropeptide of the protease is equipped at the 3′ end, as applicable, with a nucleic acid coding for a variable linker (L linker; length 0 to 40 amino acids), and cloned into a plasmid (the screening plasmid) that contains a promoter for expression of the constructs to be screened, and is optionally equipped with a resistance gene. Located in the 3′ direction from the nucleic acid coding for the prepropeptide or, if present, in the 3′ direction from the nucleic acid coding for the L linker and adjoining the nucleic acid coding for the prepropeptide, are one or more restriction enzyme interfaces to allow, by blunt end cloning, incorporation of the circular permutation mutants obtained according to the method described above, and subsequent expression thereof.

The length of the L linker depends on the distance between the new C-terminus of the mature protease and the positioning, necessary for correct processing of the preproprotein, of the prepropeptide with reference to the new C-terminus. It has been found according to the present invention, surprisingly, that the L linker can as a rule be omitted, since processing of the preproprotein as a rule proceeds successfully despite the modified position of the prepropeptide.

The individual method steps are presented once again below as an overview.

Method Steps:

1. Plasmid Restriction
The initial plasmid is digested with the corresponding enzymes in accordance with the enzyme manufacturer's instructions, in order to obtain the gene coding for the mature protein, flanked by the nucleic acids coding for the P linker fragments.

2. Agarose Gel Electrophoresis and Gel Elution
The digest is separated in an agarose gel and the desired bands are then cut out. Gel elution is performed using a kit, in accordance with the seller's protocol.

3. DNA Circularization Using T4 Ligase
The ligation mixture is prepared in ligase buffer using a DNA concentration from 1 to 5.5 ng/μL, and started by adding 1 to 40 units of ligase.

4. Ethanol Precipitation
An ethanol precipitation is performed in order to reduce the volume.

5. Remaining Linear DNA Digested Using Exonuclease III
A digest using Exonuclease III is performed at 37° C. in order to break down the linear DNA that is still present.

6. QIAquick Purification
The sample is rebuffered using the PCR purification protocol of the QIAquick purification kit (Qiagen, Hilden, Germany).

7. Partial Digest with dNase I, Stopped by Adding EDTA, to Linearize the Circularized Gene
The DNA is randomly linearized by treating it with highly dilute DNase I at room temperature.

8. QIAquick Purification
The sample is rebuffered using the PCR purification protocol.

9, Linearized DNA Repaired Using T4 DNA Polymerase and T4 DNA Ligase with Addition of dNTPs
Because perfect blunt ends are not always produced during the DNase I digest, they are generated in this step.

10. Agarose Gel Electrophoresis and Gel Elution
The digestion product is separated in an agarose gel, and the bands of the desired size are cut out. Gel elution is carried out with a kit according to protocol.

11. Clone and Screen the Linearized DNA
The linearized DNA is cloned into a prepared screening vector and transformed into a protease-negative Bacillus strain. A screening for protease activity is carried out using the resulting colonies.

The reader is further referred to the following citations regarding performance of the circular permutation method:

Qian, Z., Lutz, S. (2005) Journal of the American Chemical Society 127(39), pp. 13466-13467; Beernink et al. (2001) Protein Science, 10 (3), pp. 528-537; Baird et al. (1999) Proceedings of the National Academy of Sciences of the United States of America, 96 (20), pp. 11241-11246; Graf, R., Schachman, H. K. (1996) Proceedings of the National Academy of Sciences of the United States of America, 93 (21), pp. 11591-11596.

### Example 2

### Circular Permutation of BLAP Protease Using a Linker Made Up of 14 Amino Acids

The gene of the mature BLAP protease was used to carry out the circular permutation; a linker having a length of 14 amino acids, with the sequence GAATSGKLNGSTAG (SEQ ID NO: 1) was used as a linker to bring the N- and C-termini. The flanked construct depicted below was obtained in this fashion (what is depicted is not the DNA itself but the protein sequence corresponding to the DNA):

(SEQ ID NO: 7)

LNGSTAG [symbol] VQAPAAHNRGLTGSGVKVAVLDTGISTHPDLNI
RGGASFVPGEPSTQDGNGHGTHVAGTIAALNNSIGVLGVAPSAELYAVKV
LGADGRGAISSIAQGLEWAGNNGMHVANLSLGSPSPSATLEQAVNSATSR
GVLVVAASGNSGASSISYPARYANAMAVGATDQNNNRASFSQYGAGLDIV
APGVNVQSTYPGSTYASLNGTSMATPHVAGAAALVKQKNPSWSNVQIRNH
LKNTATSLGSTNLYGS [symbol] GAATSGK

The two linker fragments are underlined. The beginning and end of the mature protein are enclosed in boxes.

Once the circular permutation had been carried out, the two circular permutation variants NHT04240353 and NHT04241867 depicted below, among others, were obtained, and were then tested for their washing performance.

Permutation variant NHT04240353:

(SEQ ID NO: 5)

GEPSTQDGNGHGTHVAGTIAALNNSIGVLGVAPSAELYAVKVLGADGRGA
ISSIAQGLEWAGNNGMHVANLSLGSPSPSATLEQAVNSATSRGVLVVAAS
GNSGASSISYPARYANAMAVGATDQNNNRASFSQYGAGLDIVAPGVNVQS
TYPGSTYASLNGTSMATPHVAGAAALVKQKNPSWSNVQIRNHLKNTATSL
GSTNLYGS [symbol] GAATSGKLNGSTAG [symbol] VQAPAAH
NRGLTGSGVKVAVLDTGISTHPDLNIRGGASFVP

Permutation variant NHT04241867:

(SEQ ID NO: 6)

YASLNGTSMATPHVAGAAALVKQKNPSWSNVQIRNHLKNTATSLGSTNLY
GS [symbol] GAATSGKLNGSTAG [symbol] VQAPAAHNR
GLTGSGVKVAVLDTGISTHPDLNIRGGASFVPGEPSTQDGNGHGTHVAGT
IAALNNSIGVLGVAPSAELYAVKVLGADGRGAISSIAQGLEWAGNNGMHV
ANLSLGSPSPSATLEQAVNSATSRGVLVVAASGNSGASSISYPARYANAM
AVGATDQNNNRASFS QYGAGLDIVAPGVNVQSTYPGST

### Example 3

### Washing Experiments Using Circular Permutation Variant NHT04240353

The following stains were tested in order to check the washing performance of the NHT04240353 clone:
blood/milk/ink on cotton: product no. C5 of CFT B.V., Vlaardingen, Holland
whole egg/pigment on cotton: product no. 10N obtainable from wfk Testgewebe GmbH, Brüggen-Bracht, Germany, cut into small pieces
chocolate milk/ink on cotton: product no. C3 of CFT B.V., Vlaardingen, Holland
peanut oil/pigment/ink on polyester/cotton: product no. PC10 of CFT B.V., Vlaardingen, Holland
grass on cotton: product no. 164 obtainable from Eidgenössische Material- und Prüfanstalt (EMPA) Testmaterialien AG [Swiss federal materials and testing agency testing materials], St. Gallen, Switzerland
cocoa on cotton: product no. 112 obtainable from Eidgenössische Material- und Prüfanstalt (EMPA) Testmaterialien AG, St. Gallen, Switzerland

The circular permutation variant was incorporated into the baseline formulation presented below, and its washing performance was checked as compared with the initial BLAP enzyme.

TABLE 1
Baseline formulation for a textile washing agent

| Chemical name | wt % pure substance |
|---|---|
| Xanthan gum | 0.3 to 0.5 |
| Antifoaming agent | 0.2 to 0.4 |
| Glycerol | 6 to 7 |
| Ethanol | 0.3 to 0.5 |
| FAEOS | 4 to 7 |
| Nonionic surfactant | 24 to 28 |
| (FAEO, APG, etc.) | |
| Boric acid | 1 |
| Sodium citrate × 2H₂O | 1 to 2 |
| Caustic soda | 2 to 4 |
| Coconut fatty acid | 14 to 16 |
| HEDP | 0.5 |
| PVP | 0 to 0.4 |
| Optical brightener | 0 to 0.05 |
| Dye | 0 to 0.001 |
| Perfume | 0 to 2 |
| H₂O, demineralized | remainder |

Test mixture (1 ml) in 48-well plates:

| Volume | | Solution |
|---|---|---|
| 420 | μl | 161 to 966 mg textile washing agent |
| | | in 42 ml water or buffer |
| 30 to 530 | μl | 1 to 100 U/ml protease |

| | |
|---|---|
| remainder | H₂O |
| | stain diameter = 1 cm |

Incubation: 60 min, 40° C., approx. 600 rpm.

After incubation: rinse stains (3 times), dry and fix. Brightness measurement using CM508d colorimeter (Minolta).

For performance of this test, round pieces of the test fabric (diameter 10 mm) were incubated in a 24-well microtitration plate in 1 ml washing liquor for 30 minutes at 37° C. and a shaking frequency of 100 rpm. Each experiment was carried out as a triple determination.

After washing, the whiteness of the washed textiles was measured by comparison with a white standard (d/8, 8 mm, SCI/SCE) that was normalized to 100% (determination of L value). The measurement was performed on a colorimeter (Minolta CM508d) using a 10°/D65 illuminant setting. The results obtained were indicated as a percentage performance, the difference in remission values between the baseline washing agent without enzymes and the one having WT protease being normalized to 100%.

It was discovered in this fashion that the NHT04240353 variant exhibits, surprisingly, a cleaning performance in terms of whole egg/carbon black and cocoa that is approximately 50% better than the wild type BLAP, while cleaning performance in terms of grass and milk/oil is approximately as good as that of the wild type, and cleaning performance in terms of blood/milk/ink and chocolate milk/carbon black is somewhat poorer than that of the wild type.

### Example 4

### Localization of the Loops of Specific Proteases

a) Loops of BLAP (Bacillus lentus alkaline protease)

The loops are located in the following sequence segments:

Ala 1-Val 4, Arg 10-Pro 14, Asn 18-Val 26, Asp 32-Gly 45, Phe 49-His 62, Ile 70-Ser 76, Gly 78-Glu 87, Val 93-He 102, Asn 115-His 118, Leu 122-Ala 131, Ser 142-Leu 146, Ser 154-Met 169, Asp 175-Asn 179, Ala 181-Phe 183, Gly 185-He 192, Gly 196-Val 199, Tyr 203-Thr 207, Leu 211-Ser 215, Lys 231-Asn 237, Thr 247-Ala 264, and Ala 267-Thr 268.

The amino acid sequence of BLAP is as follows:

(SEQ ID NO: 1)

AQSVPWGISRVQAPAAHNRGLTGSGVKVAVLDTGISTHPDLNIRGGASFV
PGEPSTQDGNGHGTHVAGTIAALNNSIGVLGVAPSAELYAVKVLGADGRG
AISSIAQGLEWAGNNGMHVANLSLGSPSPSATLEQAVNSATSRGVLVVAA
SGNSGASSISYPARYANAMAVGATDQNNNRASFSQYGAGLDIVAPGVNVQ
STYPGSTYASLNGTSMATPHVAGAAALVKQKNPSWSNVQIRNHLKNTATS
LGSTNLYGS GLVNAEAATR

### b) Loops of BPN′

The loops are located in the following sequence segments:

Ala 1-Gly 7, Gln 10-Pro 14, Gln 19-Val 26, Asp 32-Gly 46, Phe 50-His 64, Val 95-Tyr 104, Asn 117-Asp 120, Met 124-Ala 133, Ser 145-Val 148, Glu 156-He 175, Asp 181-Gln 185, Ala 187-Phe 189, Ser 191-Val 198, Gly 202-He 205, Leu 209-Lys 213, Lys 217-Thr 220, Lys 237-Asn 243, Asn 252-Val 270, and Ala 273-Gln 275.

The amino acid sequence of BPN′ is as follows:

(SEQ ID NO: 2)

AQSVPYGVSQIKAPALHSQGYTGSNVKVAVIDSGIDSSHPALKVAGGASF
VPSETNPFQDNNSHGTHVAGTVLAVAPSASLYAVKVLGADGSGQYSWIIN
GIEWAIANNMDVINMSLGGPSGSAALKAAVDKAVASGVVVVAAAGNEGTS
GSSSTVGYPGKYPSVIAVGAVDSSNQRASFSSVGPELDVMAPGVSIWSTL
PGNKYGAKSGTCMASPHVAGAAALILSKHPNWTNTQVRSSLENTTTKLGD
SFYYGKGLINV EAAAQ

c) Loops of subtilisin Carlsberg/P300:

The loops are located in the following sequence segments:

Ala 1-Gly 7, Pro 9-Ala 13, Gln 19-Val 26, Asp 32-Gly 46, Phe 50-His 64, Val 72-Ser 89, Val 95-Tyr 104, Thr 116-Val 121, Met 124-Ala 134, Arg 145-Val 148, Ser 156-He 175, Asp 181-Asn 185, Ala 187-Phe 189, Ser 191-Val 198, Gly 202-Val 205, Tyr 209-Thr 213, Leu 217-Thr 220, Lys 237-Ala 243, Leu 250-Val 270, and Ala 274-Gln 275.

The amino acid sequence of subtilisin Carlsberg is as follows:

(SEQ ID NO: 3)

AQTVPYGIPLIKADKVQAQGFKGANVKVAVLDTGIQASHPDLNVVGGASF
VAGEAYNTDGNGHGTHVAGTVAALDNTTGVLGVAPSVSLYAVKVLNSSGS
GSYSGIVSGIEWATTNGMDVINMSLGGASGSTAMKQAVDNAYARGVVVVA
AAGNSGNSGSTNTIGYPAKYDSVIAVGAVDSNSNRASFSSVGAELEVMAP
GAGVYSTYPTNTYATLNGTSMASPHVAGAAALILSKHPNLSASQVRNRLS
STATYLGSSF YYGKGLINVEAAAQ

1-23. (canceled)

24. A polypeptide selected from the group consisting of
a) a circular permutation variant of a mature protein of a protein expressed naturally as at least one of a preprotein, proprotein, or preproprotein,
b) a variant of the circular permutation variant of (a) having sequence homology or sequence identity of at least 80% with respect to the sequence of the circular permutation variant of (a), inclusive or exclusive of the sequence of a bridging linker,
c) a variant of the circular permutation variant of (a) having at least one of a substitution, insertion, deletion, or inversion of up to 30 amino acids with reference to the polypeptide sequence of the circular permutation variant of (a), inclusive or exclusive of the sequence of a bridging linker,
d) a polypeptide that encompasses the protein variant of (a), (b), or (c).

25. The polypeptide of claim 24, wherein the protein expressed naturally as at least one of a preprotein, proprotein, or preproprotein is an enzyme.

26. The polypeptide of claim 25, wherein the enzyme is a protease.

27. The polypeptide of claim 26, wherein the protease is an alkaline protease of the subtilisin type.

28. The polypeptide of claim 27, wherein the alkaline protease of the subtilisin type is selected from the group consisting of BLAP, BPN′, subtilisin Carlsberg, and homologs thereof.

29. The polypeptide of claim 28, wherein the circular permutation variant of BLAP is selected from a polypeptide having a sequence homology or identity of at least 80% with the polypeptide of SEQ ID NO: 5 or of SEQ ID NO: 6 or from a polypeptide having at least one of an insertion, deletion, or inversion of up to 30 amino acids with reference to the polypeptide having the polypeptide sequence of SEQ ID NO: 5 or of SEQ ID NO: 6.

30. The polypeptide of claim 24, wherein the original termini of the mature protein are bridged to one another by a linker having a length of from 1 to 40 amino acids.

31. The polypeptide of claim 24, wherein new termini of the circular permutation variant are at least 10 amino acids distant from the original termini.

32. The polypeptide of claim 24, wherein new termini of the circular permutation variant are located in a region of a loop of the initial protein.

33. The polypeptide of claim 24, wherein new termini of the circular permutation variant are not located in a region of a loop of the initial protein.

34. A method for manufacturing a circular permutation variant of a protein expressed naturally as at least one of a preprotein, proprotein, or preproprotein, comprising the following steps:
a) carrying out a circular permutation method with DNA of a mature protein;
b) incorporating the DNA obtained from the circular permutation method of (a) into a suitable vector in the 3′ direction with respect to the DNA coding for at least one of a signal peptide, a propeptide and a prepropeptide;
c) introducing the vector resulting step from (b) into a suitable host for expression of the DNA, and optionally, purifying the protein obtained.

35. A polynucleotide selected from the group consisting of
a) a polynucleotide coding for a circular permutation variant of a mature protein of a protein expressed naturally as at least one of a preprotein, a proprotein or a preproprotein;
b) a mutant of the polynucleotide of (a) having a sequence homology or sequence identity of at least 80% with respect to the polynucleotide of (a), inclusive or exclusive of the sequence coding for a bridging linker;
c) a mutant of the polynucleotide of (a) having at least one of a substitution, insertion, deletion, or inversion of up to 50 nucleotides with reference to the polynucleotide sequence of a polynucleotide in accordance with (a), inclusive or exclusive of the sequence coding for a bridging linker;
d) a polynucleotide comprising the polynucleotide of (a), (b) or (c);
e) a polynucleotide that codes for the polypeptide of claim 24;
f) a polynucleotide that is complementary to the polynucleotide of (a), (b), (c), (d) or (e).

36. The polynucleotide of claim 35, wherein the protein expressed naturally as at least one of a preprotein, proprotein or preproprotein of (a) is an enzyme.

37. The polynucleotide of claim 36, wherein the enzyme is a protease.

38. The polynucleotide of claim 37, wherein the protease is an alkaline protease of the subtilisin type.

39. The polynucleotide of claim 38, wherein the alkaline protease of the subtilisin type is selected from the group consisting of BLAP, BPN′, and subtilisin Carlsberg.

40. The polynucleotide of claim 39, coding for a circular permutation variant of BLAP selected from a polypeptide having a sequence homology or sequence identity of at least 80% with the polypeptide of SEQ ID NO: 5 or of SEQ ID NO: 6.

41. A vector comprising the polynucleotide of claim 35.

42. An agent comprising the polypeptide of claim 24, wherein the agent is selected from the group consisting of a washing agent and a cleaning agent.

43. An agent comprising the polypeptide of claim 24, wherein the agent is selected from the group consisting of a cosmetic preparation and a pharmaceutical preparation.
